(19) United States
(12) Patent Application Publication
(10) Pub. No.: US 2003/0135196 A1
(43) Pub. Date: Jul. 17, 2003

(54) Treatment of neurologic hemorrhage

(75) Inventors: David P. Hesson, Malvern, PA (US); Glen D. Frazer, Wynnewood, PA (US); Timothy J. Pelura, Malvern, PA (US); Barbara L. Powers, Phoenixville, PA (US); Bruce Shook, Devon, PA (US)

Correspondence Address: ALLEN BLOOM, C/O DECHERT, PRINCETON PIKE CORPORATION CENTER, P.O. BOX 5218, PRINCETON, NJ, 08543-5218, US

(73) Assignee: Neuron Therapeutics, Inc., Malvern, PA (US)

(21) Appl. No.: 10/229,619

(22) Filed: Aug. 28, 2002

Related U.S. Application Data
(60) Provisional application No. 60/316,235, filed on Aug. 31, 2001.
(60) Provisional application No. 60/347,398, filed on Jan. 10, 2002.

(51) Int. Cl. A61M 31/00

(52) U.S. Cl. 604/500; 424/531

(57) ABSTRACT

Subarachnoid hemorrhage is treated by circulating a synthetic cerebrospinal fluid containing an emulsifying agent in the cerebrospinal fluid pathway in the vicinity of hemorrhage. The emulsifying agent scavenges blood products and circulation of the synthetic cerebrospinal fluid clears the blood products form the subarachnoid space to reduce cerebral vasospasm.

This application claims the priority of U.S. Provisional Applications No. 60/316,235, filed Aug. 31, 2001 and No. 60/347,398, filed Jan. 10, 2002, both of which are incorporated herein by reference in their entirety.

This invention is in the field of neurosurgery, and in particular, it relates to using a synthetic cerebrospinal fluid to clear blood products, inflammatory products and tissue debris from the space surrounding neural tissue such as the subarachnoid spaces

Neurologic hemorrhage is characterized as bleeding into spaces surrounding the neurologic tissue of the brain and spinal cord. One example of this phenomenon, subarachnoid hemorrhage (SAH), is characterized by bleeding into the subarachnoid space surrounding the brain. Subarachnoid bleeding results from processes such as head trauma, coagulation disorders, ruptured aneurysms and arteriovenous malformations. Aneurysmal SAH is the most common cause of non-traumatic SAH, accounting for 75%-85% of all cases. Subarachnoid blood can also originate from intraventricular or intracerebral hemorrhages.

A significant complication of SAH and other neurologic bleeding is cerebral vasospasm, which is defined as the delayed narrowing of major cerebral arteries. The incidence of symptomatic vasospasm is 25-30% after aneurysmal SAH. Symptomatic vasospasm leads to the development of focal or global neurological deficits. Approximately 50% of patients with symptomatic vasospasm will develop ischemic stroke. Fifteen to 20% of patients with symptomatic vasospasm will die from progressive cerebral ischemia.

Although many risk factors for the development of vasospasm have been identified, the most significant predictor is the amount and location of subarachnoid blood. There is a direct correlation between the amount of subarachnoid blood visualized on CT scan and risk of vasospasm. Patients with clot in the basal cisterns or clot greater than 1 mm in thickness are also more likely to develop vasospasm.

Data from several investigations show that compounds from hemolyzed erythrocytes initiate the vasospastic process. In particular, hemoglobin and its breakdown products have been identified as the primary etiological agents. Other blood products that may contribute to vasospasm are platelets, fibrin degradation products, catecholamines, histamine, thrombin, plasmin, serotonin, and eicosanoids.

Vasospasm develops 3-14 days after hemorrhage and typically peaks within 5-7 days. Thus, a window of opportunity is available to remove subarachnoid blood and prevent or reduce the severity of disabling cerebral ischemia. Blood and clots can be removed by surgical debridement, but this procedure often results in only partial clot removal and risks of further bleeding and ischemic stroke. Thrombolytics such as urokinase and tissue plasminogen activator (tPA) can be injected into the ventricular system to dissolve clots, but this treatment also increases the risk of additional hemorrhage. Other pharmacological agents used intravenously such as nimodipine (calcium antagonist), glucocorticoid steroids, nonsteroidal anti-inflammatories, cyclosporine A (an antiinflammatory), reserpine (an antihypertensive), and kanamycin do not consistently provide protection against vasospasm.

The prior art discloses treatments for subarachnoid hemorrhage. U.S. Pat. No. 4,792,564 to Harder et al. teaches a method to treat cerebral vasospasm from subarachnoid hemorrhage by intravenously infusing nicorandil. U.S. Pat. No. 5,648,351 to Kelly et al. describes oral, parenteral, and intramuscular administration of a macrolide to treat cerebral ischemia after subarachnoid hemorrhage. U.S. Pat. No. 4,393,863 to Osterholm discloses an oxygenated synthetic cerebrospinal fluid that treats ischemic tissue. U.S. Pat. No. 4,904,237 to Janese describes an apparatus that detects blood in the cerebrospinal fluid and teaches a method to exchange native cerebrospinal fluid with filtered, native cerebrospinal fluid. Kodama et al. (Surg. Neurol. 53:110-118, 2000) describe cisternal irrigation with lactated Ringer's solution at rates of 0.5 mL/min (30 mL/hr) over 2-18 days to treat SAH.

Another complication of neurologic bleeding and trauma are neurologic adhesions, where adjacent tissue or structures within the CNS adhere to each other. The neurologic trauma giving rise to the adhesion can be the result of conditions such as tethered cord syndrome (TCS; see S. Huttman et al., “Surgical Management of Tethered Spinal Cord in Adults: Report of 54 Cases.”, J. Neurosurg. 95(2 Suppl):173-178, 2001), spinal adhesive arachnoiditis (inflammation of the arachnoid membrane that forms the intermediate membrane of the meninges; see, R. Dolan, “Spinal adhesive arachnoiditis.” Surg. Neurol. 39(6):479-484, 1993) and syringomyelia (see, Ohata et al. J. Clin. Neurosci. 8(1):40-42, 2001). Neurologic adhesions can result in pain, lack of mobility and further trauma when then adjacent structures pull against each other. This may result in continuing or increasing damage to the effected area.

Thus, as described above, various therapeutics and techniques have been proposed to treat subarachnoid hemorrhage. Notwithstanding these prior therapeutic agents and techniques, new treatments having the features of the present invention are needed.

### SUMMARY OF THE INVENTION

The present invention is a method of treating neurologic hemorrhage and trauma, including for example hemorrhage indications such as subarachnoid hemorrhage (SAH), intraventricular hemorrhage (IVH), intracerebral hemorrhage (ICH), spinal cord hemorrhage (SCH), or neurologic adhesions, using a synthetic cerebrospinal fluid, which can be administered at elevated flow rates. The synthetic cerebrospinal fluid scavenges blood products in the subarachnoid space and ventricles. Clearance of these products from the CSF pathway decreases the risk of developing cerebral vasospasm and neurologic tissue adhesions.

In one embodiment, therapeutic agents are added to the synthetic cerebrospinal fluid, which can be done after an initial clearing of the CSF pathway. After administration of the therapeutic agents by perfusion, synthetic cerebrospinal fluid can again be perfused without the therapeutic agent.

The synthetic CSF comprises and artificial cerebrospinal aqueous fluid containing physiologic, with reference to the central nervous system, amounts of electrolytes such as Na⁺, K⁺, Ca⁺², Mg⁺², PO₄⁻², and HCO₃⁻ balanced at the proper pH. In addition the synthetic CSF can contain, a sugar such as dextrose, and/or an oxygen carrying fluorocarbon with an emulsifying agent, and/or an oncotic agent such as a protein like human serum albumin, and/or amino acids. The components of the synthetic CSF are similar to those found in natural CSF. The fluorocarbon is usually a perfluorocarbon or a highly fluorinated hydrocarbon that is dispersed in the aqueous synthetic CSF with the aid of a biocompatible surfactant. The synthetic CSF can also contain agents to dissolve clots such as tissue plasminogen activator (tPA) or urokinase, or agents to decrease free radical formation such as ascorbic acid or alpha-ketoglutaric acid.

The human serum albumin, as a component of the synthetic CSF, scavenges blood products, surgical debris and metabolic toxins while also acting as an oncotic agent, which in turn helps support elevated perfusion rates such as 2-80 or 5-80 mL/min. Such elevated flow rates allow rapid removal of blood and blood components from neural tissue, thus limiting damage and decreasing the risk to the patient. Therapeutic agents such as thrombolytics, vasodilating drugs, antiinflammatory drugs, antioxidants and anti-adhesion agents can be included in the synthetic CSF to also prevent, inhibit or ameliorate the development of cerebral vasospasm and adhesions.

In one embodiment, provided is a method of treating neurologic hemorrhage comprising: inserting a inflow catheter and an outflow catheter into a cerebrospinal pathway to create a flow pathway for irrigating the area of the hemorrhage; and irrigating via the flow pathway the area of the hemorrhage with a synthetic cerebrospinal fluid for a period of time effective to reduce an indication of the presence of blood in effluent from the outflow catheter.

In one embodiment, provided is a method of reducing the occurrence of or ameliorating the severity of neurologic adhesion resulting from a surgery on cerebrospinal tissue or from an inflammation of cerebrospinal tissue comprising: identifying a subject with a tissue at risk for forming a neurologic adhesion resulting from a surgery on cerebrospinal tissue or from an inflammation of cerebrospinal tissue; inserting a inflow catheter and an outflow catheter into a cerebrospinal pathway to create a flow pathway for irrigating the tissue; and irrigating via the flow pathway the tissue with a synthetic cerebrospinal fluid, wherein, in the case of surgery the synthetic cerebrospinal fluid does not have a respiration-supporting amount of oxygen, and wherein the inflammation of cerebrospinal tissue does not result from stroke.

For example, the irrigating in such methods is maintained for at least, in aggregate, 6 hours. In one embodiment, such methods further comprise surgically removing a subarachnoid clot in conjunction with the other steps. In one embodiment of such methods, the synthetic cerebrospinal fluid is infused into the subarachnoid space of the brain, and drained from the spinal subarachnoid space. In another embodiment of such methods, the irrigating is initiated prior to, or concurrently with, initiating an operation to repair an aneurysm.

### BRIEF DESCRIPTION OF THE DRAWINGS

FIGS. 1 and 2 display schematically a flow apparatus for flow through the cerebral spinal pathway.

FIG. 3 shows how the patient can be inclined during administration to achieve high flow rates.

FIG. 4 shows an efflux flow rate control mechanism.

FIG. 5 shows a pressure break device.

FIG. 6 illustrates another device for controlling pressure at the drainage end.

### DETAILED DESCRIPTION OF THE INVENTION

Subarachnoid hemorrhage often results form a ruptured aneurysm in the Circle of Willis. Cerebral vessels can constrict when exposed to the leaking blood and blood degradation products in the subarachnoid space. As used herein, the term “blood product” includes blood cells (including for example lymphocytes and platelets), blood components such as polypeptides and other chemicals chemical complexes, and blood breakdown products of the foregoing that can accumulate in the cerebrospinal fluid (hereafter “CSF”) following neurologic hemorrhage such as SAH, IVH, ICH or SCH. These blood products can include, for example, hemoglobin, hemoglobin degradation products, products of lipid peroxidation, and hemostatic and anticoagulant factors.

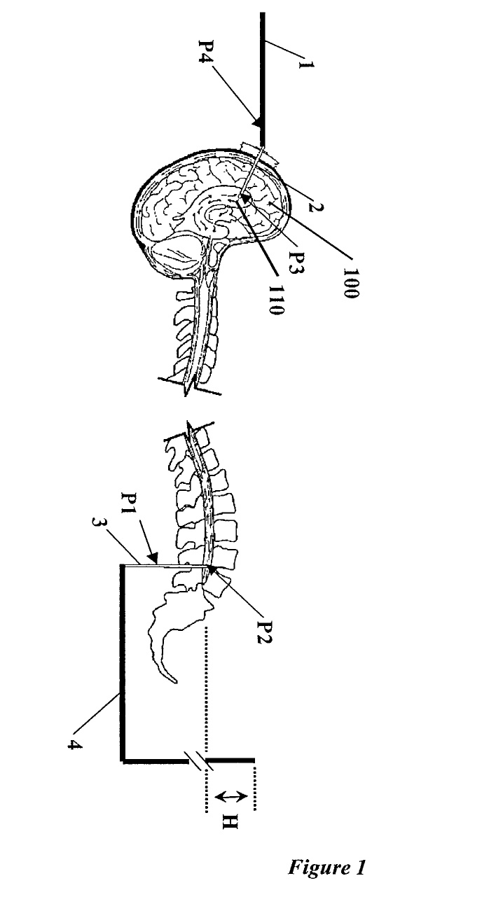

As seen in FIG. 1, the present invention is a method to treat subarachnoid hemorrhage by removing blood products from the subarachnoid space 100. A synthetic cerebrospinal fluid can be infused into the cerebrospinal fluid pathway at a point such as the lateral ventricles 110, and withdrawn from a variety of locations in the cerebrospinal fluid pathway. Any pathway through neurologic tissue spaces having an entrance into and an exit from that space which space contains blood products, is within the scope of the invention. Suitable catheters used to deliver and drain the synthetic CSF can be similar to those disclosed in U.S. application Ser. No. 09/382,136, filed Nov. 26, 1999, the entirety of which are incorporated herein by reference.

Subarachnoid hemorrhage can be identified on a non-contrast head CT with almost 100% accuracy. CSF in the ventricles (such as lateral ventricles 110) and subarachnoid space 100 is nearly black (low attenuation), while blood in those spaces is white (high attenuation) on the scan. Subarachnoid hemorrhage can also be detected by lumbar puncture. CSF samples obtained by this method contain frank blood or high red cell counts as well as increased protein concentrations.

Subarachnoid hemorrhage is treated according to the present invention with a physiologic synthetic cerebrospinal fluid capable removing blood from the subarachnoid space. The fluid is capable of cleansing the subarachnoid space and scavenging blood and blood products which can lead to vasospasm.

Artificial Cerebrospinal Aqueous Fluid

The artificial cerebrospinal aqueous fluid is comprised of components which approach many of the physical and chemical characteristics of natural cerebrospinal fluid while maintaining its ability to bind blood products. Accordingly, the fluid preferably contains amounts of sodium, potassium, magnesium, calcium, chloride, and bicarbonate ions in amounts approximating those of natural cerebrospinal fluid, and (in a preferred embodiment) an oncotic agent such as, for example, human serum albumin (see table below). Similarly, the fluid can contain amino acids, amino acid precursors, glucose, and other moieties normally found in natural cerebrospinal fluid.

Exemplary Formulation 1

| Component | Amount Per Dose | |
|---|---|---|
| Oxygen Carrying Fluorocarbon | 0 or 5-20% | w/v |
| Component | | |
| Na⁺ | 135-145 | mEq/L |
| HCO₃⁻ | 20-25 | mEq/L |
| P0₄⁻² | 1.2-2.0 | mg/L |
| K⁺ | 2.7-3.9 | mEq/L |
| Mg⁺² | 2.0-2.5 | mEq/L |
| Ca⁺² | 2.0-3.0 | mEq/L |
| Cl⁻ | 115-125 | mEq/L |
| Glucose | 0 or 500-1500 | mg/L |
| Albumin | 5-40 | g/L |
| | (or 10-30 | g/L) |

Exemplary Formulations 2-6

| | # 2 | | # 3 | | # 4 | | # 5 | | # 6 | |
|---|---|---|---|---|---|---|---|---|---|---|
| | Quantity/ | | Quantity/ | | Quantity/ | | Quantity/ | | Quantity/ | |
| Component | 2400 mL | | 2138 mL | | 2138 mL | | 2138 mL | | 2138 mL | |
| F44E | 400 | g | — | | — | | — | | — | |
| NaCl, USP | 15.3 | g | 15.3 | g | 15.3 | g | 15.3 | g | 15.3 | g |
| NaHCO₃, USP | 4.14 | g | 4.14 | g | 4.14 | g | 4.14 | g | 4.14 | g |
| KCl, USP | 0.46 | g | 0.46 | g | 0.46 | g | 0.46 | g | 0.46 | g |
| MgCl₂-6H₂O, USP | 0.48 | g | 0.48 | g | 0.48 | g | 0.48 | g | 0.48 | g |
| CaCl₂-2H₂O, USP | 0.36 | g | 0.36 | g | 0.36 | g | 0.36 | g | 0.36 | g |
| Egg Yolk Phospholipid | 27.9 | g | — | | — | | — | | — | |
| Dextrose, USP | 2 | g | 2 | g | 2 | g | 2 | g | 2 | g |
| Albumin (Human), USP | 40 | g | 40 | g | 20 | g | 60 | g | 40 | g |
| L-lysine HCl, USP | 6.48 | mg | 6.48 | mg | 6.48 | mg | 6.48 | mg | — | |
| L-alanine, USP | 6.86 | mg | 6.86 | mg | 6.86 | mg | 6.86 | mg | — | |
| L-serine, USP | 5.90 | mg | 5.90 | mg | 5.90 | mg | 5.90 | mg | — | |
| L-threonine,USP | 7.10 | mg | 7.10 | mg | 7.10 | mg | 7.10 | mg | — | |
| L-arginine, USP | 4.48 | mg | 4.48 | mg | 4.48 | mg | 4.48 | mg | — | |
| L-leucine, USP | 2.96 | mg | 2.96 | mg | 2.96 | mg | 2.96 | mg | — | |
| L-isoleucine, USP | 1.24 | mg | 1.24 | mg | 1.24 | mg | 1.24 | mg | — | |
| L-valine, USP | 3.98 | mg | 3.98 | mg | 3.98 | mg | 3.98 | mg | — | |
| L-phenylalanine, USP | 1.98 | mg | 1.98 | mg | 1.98 | mg | 1.98 | mg | — | |
| L-tyrosine, USP | 1.90 | mg | 1.90 | mg | 1.90 | mg | 1.90 | mg | — | |
| L-histidine, USP | 2.46 | mg | 2.46 | mg | 2.46 | mg | 2.46 | mg | — | |
| L-methionine, USP | 0.50 | mg | 0.50 | mg | 0.50 | mg | 0.50 | mg | — | |
| NaH₂PO₄, USP | 8.20 | mg | 8.20 | mg | 8.20 | mg | 8.20 | mg | 8.20 | |
| Na₂HPO₄, USP | 1.22 | mg | 1.22 | mg | 1.22 | mg | 1.22 | mg | 1.22 | mg |
| α-ketoglutaric acid | 60 | mg | 60 | mg | 60 | mg | 60 | mg | 60 | mg |
| Water for lnjection, USP | 2081 | mL | 2081 | mL | 2081 | mL | 2081 | mL | 2081 | mL |

Exemplary Formulations 7-11

| | # 7 | | # 8 | | # 9 | | # 10 | | # 11 | |
|---|---|---|---|---|---|---|---|---|---|---|
| | Quantity/ | | Quantity/ | | Quantity/ | | Quantity/ | | Quantity/ | |
| Component | 2138 mL | | 2138 mL | | 2138 mL | | 2138 mL | | 2138 mL | |
| NaCl | 15.3 | g | 15.3 | g | 15.3 | g | 15.3 | g | 15.3 | g |
| NaHCO₃ | 4.14 | g | 4.14 | g | 4.14 | g | 4.14 | g | 4.14 | g |
| KCl | 0.46 | g | 0.46 | g | 0.46 | g | 0.46 | g | 0.46 | g |
| MgCl₂-6H₂O | 0.48 | g | 0.48 | g | 0.48 | g | 0.48 | g | 0.48 | g |
| CaCl₂-2H₂O | 0.36 | g | 0.36 | g | 0.36 | g | 0.36 | g | 0.36 | g |
| Dextrose | — | | 2 | g | 2 | g | 2 | g | 2 | g |
| Albumin (Human) | 40 | g | 40 | g | 40 | g | 40 | g | 40 | g |
| NaH₂PO₄ | 8.20 | mg | 8.20 | mg | 8.20 | mg | 8.20 | mg | 8.20 | mg |
| Na₂HPO₄ | 1.22 | mg | 1.22 | mg | 1.22 | mg | 1.22 | mg | 1.22 | mg |
| α-Ketoglutaric acid | — | | — | | — | | 60 | mg | 60 | mg |
| Ascorbic acid | — | | 8,500 | mg | 8,500 | mg | 8,500 | mg | 8,500 | mg |
| Urokinase | — | | — | | 256,000 | IU | — | | 256,000 | IU |
| Water for Injection | 2081 | mL | 2081 | mL | 2081 | mL | 2081 | mL | 2081 | mL |

Exemplary Formulations 12-16

| | # 12 | | # 13 | | # 14 | | # 15 | | # 16 | |
|---|---|---|---|---|---|---|---|---|---|---|
| | Quantity/ | | Quantity/ | | Quantity/ | | Quantity/ | | Quantity/ | |
| Component | 2138 mL | | 2138 mL | | 2138 mL | | 2138 mL | | 2138 mL | |
| NaCl | 15.3 | g | 15.3 | g | 15.3 | g | 15.3 | g | 15.3 | g |
| NaHCO₃ | 4.14 | g | 4.14 | g | 4.14 | g | 4.14 | g | 4.14 | g |
| KCl | 0.46 | g | 0.46 | g | 0.46 | g | 0.46 | g | 0.46 | g |
| MgCl₂-6H₂O | 0.48 | g | 0.48 | g | 0.48 | g | 0.48 | g | 0.48 | g |
| CaCl₂-2H₂O | 0.36 | g | 0.36 | g | 0.36 | g | 0.36 | g | 0.36 | g |
| dextrose | 2 | g | 2 | g | 2 | g | 2 | g | 2 | g |
| Albumin (Human) | 40 | g | 20 | g | 5 | g | 40 | g | 40 | g |
| NaH₂PO₄ | 8.20 | mg | 8.20 | mg | 8.20 | mg | 8.20 | mg | 8.20 | mg |
| Na₂HPO₄ | 1.22 | mg | 1.22 | mg | 1.22 | mg | 1.22 | mg | 1.22 | mg |
| α-Ketoglutaric acid | 60 | mg | 60 | mg | 60 | mg | 60 | mg | 60 | mg |
| Ascorbic acid | — | | 8,500 | mg | 8,500 | mg | 8,500 | mg | 8,500 | mg |
| Urokinase | — | | — | | — | | — | | 100,000 | IU |
| Tissue plasminogen | 100 | IU/mL | 100 | IU/mL | 100 | IU/mL | 100 | IU/mL | 50 | IU/mL |
| activator (tPA) | | | | | | | | | | |
| Water for Injection | 2081 | mL | 2081 | mL | 2081 | mL | 2081 | mL | 2081 | mL |

Exemplary Formulations 17-21

| | # 17 | | # 18 | | # 19 | | # 120 | | # 21 | |
|---|---|---|---|---|---|---|---|---|---|---|
| | Quantity/ | | Quantity/ | | Quantity/ | | Quantity/ | | Quantity/ | |
| Component | 2138 mL | | 2138 mL | | 2138 mL | | 2138 mL | | 2138 mL | |
| NaCl | 15.3 | g | 15.3 | g | 15.3 | g | 15.3 | g | 15.3 | g |
| NaHCO₃ | 4.14 | g | 4.14 | g | 4.14 | g | 4.14 | g | 4.14 | g |
| KCl | 0.46 | g | 0.46 | g | 0.46 | g | 0.46 | g | 0.46 | g |
| MgCl₂-6H₂O | 0.48 | g | 0.48 | g | 0.48 | g | 0.48 | g | 0.48 | g |
| CaCl₂-2H₂O | 0.36 | g | 0.36 | g | 0.36 | g | 0.36 | g | 0.36 | g |
| dextrose | 2 g | 2 | g | 2 | g | 2 | g | 2 | g | |
| Albumin (Human) | 40 | g | 20 | g | 5 | g | 40 | g | 40 | g |
| NaH₂PO₄ | 8.20 | mg | 8.20 | mg | 8.20 | mg | 8.20 | mg | 8.20 | mg |
| Na₂HPO₄ | 1.22 | mg | 1.22 | mg | 1.22 | mg | 1.22 | mg | 1.22 | mg |
| α-Ketoglutaric acid | 60 | mg | 60 | mg | 60 | mg | 60 | mg | 60 | mg |
| Ascorbic acid | — | | 8,500 | mg | 8,500 | mg | 8,500 | mg | 8,500 | mg |
| Urokinase | — | | — | | — | | — | | 100,000 | IU |
| Tissue plasminogen | 29,000 | IU/mL | 29,000 | IU/mL | 1,000 | IU/mL | 500 | IU/mL | 1,000 | IU/mL |
| activator (tPA) | | | | | | | | | | |
| Water for Injection | 2081 | mL | 2081 | mL | 2081 | mL | 2081 | mL | 2081 | mL |

Exemplary Formulations 22-26

| | # 22 | | # 23 | | # 24 | | # 25 | | # 26 | |
|---|---|---|---|---|---|---|---|---|---|---|
| | Quantity/ | | Quantity/ | | Quantity/ | | Quantity/ | | Quantity/ | |
| Component | 2138 mL | | 2138 mL | | 2138 mL | | 2138 mL | | 2138 mL | |
| NaCl, USP | 15.3 | g | 15.3 | g | 15.3 | g | 15.3 | g | 15.3 | g |
| NaHCO₃, USP | 4.14 | g | 4.14 | g | 4.14 | g | 4.14 | g | 4.14 | g |
| KCl, USP | 0.46 | g | 0.46 | g | 0.46 | g | 0.46 | g | 0.46 | g |
| MgCl₂-6H₂O, USP | 0.48 | g | 0.48 | g | 0.48 | g | 0.48 | g | 0.48 | g |
| CaCl₂-2H₂O, USP | 0.36 | g | 0.36 | g | 0.36 | g | 0.36 | g | 0.36 | g |
| Dextrose, USP | 2 | g | 2 | g | 2 | g | 2 | g | 2 | g |
| Albumin (Human), USP | 40 | g | 40 | g | 20 | g | 40 | g | 40 | g |
| L-lysine HCl, USP | 6.48 | mg | — | | 6.48 | mg | 6.48 | mg | — | |
| L-alanine, USP | 6.86 | mg | — | | 6.86 | mg | 6.86 | mg | — | |
| L-serine, USP | 5.90 | mg | — | | 5.90 | mg | 5.90 | mg | — | |
| L-threonine, USP | 7.10 | mg | — | | 7.10 | mg | 7.10 | mg | — | |
| L-arginine, USP | 4.48 | mg | — | | 4.48 | mg | 4.48 | mg | — | |
| L-leucine, USP | 2.96 | mg | — | | 2.96 | mg | 2.96 | mg | — | |
| L-isoleucine, USP | 1.24 | mg | — | | 1.24 | mg | 1.24 | mg | — | |
| L-valine, USP | 3.98 | mg | — | | 3.98 | mg | 3.98 | mg | — | |
| L-phenylalanine, USP | 1.98 | mg | — | | 1.98 | mg | 1.98 | mg | — | |
| L-tyrosine, USP | 1.90 | mg | — | | 1.90 | mg | 1.90 | mg | — | |
| L-histidine, USP | 2.46 | mg | — | | 2.46 | mg | 2.46 | mg | — | |
| L-methionine, USP | 0.50 | mg | — | | 0.50 | mg | 0.50 | mg | — | |
| NaH₂PO₄, USP | 8.20 | mg | 8.20 | mg | 8.20 | mg | 8.20 | mg | 8.20 | mg |
| Na₂HPO₄, USP | 1.22 | mg | 1.22 | mg | 1.22 | mg | 1.22 | mg | 1.22 | mg |
| α-ketoglutaric acid | 60 | mg | 60 | mg | 60 | mg | 60 | mg | 60 | mg |
| Tissue plasminogen | 27,000 | IU/mL | 27,000 | IU/mL | 27,000 | IU/mL | 27,000 | IU/mL | 27,000 | IU/mL |
| activator (tPA) | | | | | | | | | | |
| Ascorbic acid | — | | — | | — | | 8,500 | mg | 8,500 | mg |
| Water for Injection, USP | 2081 | mL | 2081 | mL | 2081 | mL | 2081 | mL | 2081 | mL |

When dextrose, amino acids or α-ketoglutaric acid are present, they are present in amounts selected to provide nutrients for the perfused tissue. The preferred amino acids for nutrient use are those listed above.

Fluorocarbon

The fluid can also contain one or more non-aqueous oxygen transfer compounds which are capable of dissolving gases such as oxygen and transferring the oxygen to neural tissue. Preferred oxygen transfer compounds comprise silicone polymers and fluorocarbon polymers, preferably, perfluorocarbon polymers and fluorocarbon polymers, and most preferably, t-bis-perfluorobutyl ethylene. The use of such oxygen transfer compounds in synthetic CSF is described in U.S. Pat. No. 4,981,691, which is incorporated by reference. The basic requirements for oxygen transfer compounds are effectiveness in carrying a physiologically useful amount of oxygen. Factors involved in selecting preferred such compounds include oxygen capacity, tissue retention (preferably minimized), emulsion stability, toxicity, and the like. Such compounds are described, for example, in: Riess et al., “Design Synthesis and Evaluation of Fluorocarbons and Surfactants for In vivo Applications New Perfluoroalkylated Polyhydroxylated Surfactants”, Biomat. Artif. Cells Artif. Organs, 16:421-430 (1988); Riess, Reassessment of criteria for the Selection of Perfluorochemicals for Second-Generation Blood Substitutes: Analysis of Structure/Property Relationships, Artificial Organs 8:44-56 (1984); Riess, et al., Design, Synthesis and Evaluation of Fluorocarbons and Surfactants for In Vivo Applications New Perfluoroalkylated Polyhydroxylated Surfactants, Biomat. Artif. Cells Artif. Organs 16:421-430 (1988); Riess, et al., Solubility and Transport Phenomena in Perfluorochemicals Relevant to Blood Substitution and Other Biomedical Applications, Pure & Applied Chem., 54:2383-2406 (1982); Yamanouchi, et al., Quantitative Structure-In Vivo Half-Life Relationships of Perfluorochemicals for Use as Oxygen Transporters, Chem., Pharm. Bull., 33:1221-1231 (1985); Lowe, et al., Perfluorochemicals: Blood Substitutes and Beyond Adv. Mater, 3:87-93 (February, 1991); Riess, et al., Fluorocarbon-Based In Vivo Oxygen Transport and Delivery Systems Vox Sang, 61:225-239 (December 1991); and Weers, et al., U.S. Pat. No. 5,914,352.

Among preferred poly-fluorinated, oxygen-carrying compounds are those of the formula

CₘFₘ₊₁—CH═CH—CₙFₙ₊₁,

where m+n equals 6 to 10. Preferably, the double bond is trans. One preferred polyfluorinated, oxygen-carrying compound is trans-Bis-perfluorobutyl ethylene (m and n each equal 4). Also preferred are those of the formula

CₘFₘ₊₁—O—CₙFₙ₊₁,

where m+n equals 6 to 9 (or 8). One of the perfluoro alkyls can be substituted with a halo from Br (preferably), Cl or I. Further preferred are those of the formula

CₘFₘ₊₁—R,

where m is 8 (or 10) to 12 and R is Br, Cl, I, or C₁-C₃ alkyl.

Fluorocarbon compounds are compounds, generally oils, which are substantially immiscible in and insoluble in aqueous fluids. Emulsification makes it possible to introduce these hydrophobic molecules into the natural or artificial CSF in a readily dispersible form since the resulting fluorocarbon emulsion particles will be coated with an amphipathic layer.

Fluorocarbon molecules used in these emulsions can have various structures, such as cyclic, straight or branched chain. These molecules can also have some degree of unsaturation and can also contain bromine or hydrogen atoms, or they can be amine derivatives. The fluorocarbons can be present in the emulsion in concentrations ranging from about 5% to 20% weight per volume (w/v). The preferred concentration is from 12% to 17% (w/v).

Again, when a non-aqueous oxygen transfer compound is included in the fluid, a suitable emulsifying agent can be added to facilitate dispersion of the oxygen transfer compound in the fluid and form an oxygenated aqueous emulsion of, e.g., a fluorocarbon. The inclusion of oxygen transfer compounds in the fluid is particularly desirable when it is necessary to treat hypoxic-ischemic conditions in the tissue as well as subarachnoid hemorrhage.

The emulsifying agents generally used are anionic, cationic, or nonionic surfactants, fluorosurfactants, condensates of ethylene oxide and propylene glycol, or natural amphipathic compounds such as phospholipids, particularly phosphatidylcholine, or any combination thereof. When phospholipid is used as the emulsifying agent, it is typically included in the range of from 2 to 14% w/v, where higher phospholipid concentration typically corresponds with higher fluorocarbon concentration, though particle size and the emulsion-forming character of the fluorocarbon impact on the amount of phospholipid needed. It will be recognized that when an emulsifying agent serves to emulsify a fluorocarbon, it further serves as a suspending or solubilizing agent.

Other Features

The aqueous or non-aqueous phase of the emulsion can also comprise a therapeutic agent to further treat cerebral vasospasm. Thrombolytics and vasodilators such as papaverine, adenosine, and nitroprusside can be included in the fluid, as well as iron chelators, antioxidants, calcium channel blockers, and the like. Depending on the cost of the therapeutic agent, a treating physician may elect to provide the therapeutic as a bolus amount in the administered synthetic cerebrospinal fluid, with flow shut down after the affected area can be anticipated to be bathed with a useful concentration for the therapeutic agent. This can be at the end of the irrigating procedure, or during a temporary shutdown in flow for a period of time adapted to provide a therapeutically effective exposure to the therapeutic agent. Another variation includes albumin in the synthetic cerebrospinal fluid, to scavenge blood products and, depending on the concentrations used, provide an oncotic effect to reduce tissue edema as in U.S. application Ser. No. 09/440,038, the entirety of which is herein incorporated by reference.

Methods of synthetic cerebrospinal fluid circulation are also disclosed in U.S. application Ser. No. 09/440,038. Treatment of neurologic hemorrhages can be achieved with any combination of fluid introduction and withdrawal points in the CSF pathway. Fluid introduction points can be, for example, a ventricle, a site of surgical intervention or the basal cistern. Selection of an introduction point depends on the site of the neural tissue to be treated. However, the first point used for fluid introduction is preferably one of the lateral ventricles 100. The second point in the pathway can be the ipsilateral ventral, the sulcus of Sylvius, the basal cistern or the lumbar space. The point in the pathway used for fluid withdrawal is preferably the lumbar spinal subarachnoid space 140.

After subarachnoid hemorrhage in a patient, infusion of a synthetic CSF with a blood product scavenger contacts and binds blood products in the subarachnoid space. These blood products are cleared from the CSF pathway with the withdrawal of fluid, thereby reducing patient risk of developing cerebral vasospasm.

The fluid can be circulated in a continuous, semi-continuous, intermittent, or pulsating manner. The flow rate of the synthetic CSF will typically be in the range of about 2 to 80 mL/min or preferably 5 to 80 mL/min, more preferred 10-50 mL/min, with the actual flow desirably being titrated to maintain physiologic pressure in the intrathecal space. Perfusion is typically continued for 6-72 hours until there is sufficient clearance of blood. A perfusion time of 12-48 hours is preferred.

In certain embodiments of the method, the efficacy of treatment and removal of blood can be monitored, for example by serial head CT scans, or monitoring an indication of the presence of blood (as is recognized in the art) in the effluent from the irrigation method. Ordinary experimentation can be used to identify the level of an indication of blood that provide a useful indication that the amount of subarachnoid blood as detected for example by CT scan has been reduced. Irrigating flow pursuant to the invention can be ended upon reaching a target clearance of blood.

Intra-Operative Irrigation

In one embodiment of the invention, the irrigating the area of the hemorrhage is initiated prior to or concurrently with the initiation of an operation to repair a aneurysm. Preferably, the irrigation is maintained for a period of time after the operation. In one embodiment, the irrigation is used to lower the temperature of the brain during the operation. Such temperature reduction is preferably, for a human subject, effected by irrigating a synthetic cerebrospinal fluid with a temperature from 15° C. to 35° C., more preferably from 28° C. to 33° C. After the operation, the temperature can be increased, preferably at no more than 37° C.

Adhesion Prevention

The procedures described herein can be conducted in conjunction with, or following a surgery, other trauma, or inflammation event giving rise to an increased risk of a tissue adhesion. Such surgeries include spinal cord surgeries, spinal disc surgery, and surgeries for management of syringomyelia. Surgeries which are associated with a risk of giving rise to an inappropriate tissue-to-tissue adhesion will be recognized by surgeons. Such trauma or inflammation events that can be identified to indicate a need for the treatment of the invention include syringomyclia and arachnoiditis.

While not being limited to theory, it is believed that blood components, degradation products, tissue debris or inflammation-related signaling molecules from immune cells increases the risk of an adhesion between two tissues. Thus, application of the procedures described herein can reduce the incidence of, or ameliorate the severity of, tissue adhesions in tissue in the cerebrospinal pathway.

Moreover, the irrigating composition can include a number of negatively charged polymeric agents that inhibit or ameliorate the formation of tissue adhesions (such as block copolymers, hyaluronates, and 2-hydroxyethyl starches (e.g., Hespan, DuPont, Wilmington, Del.), or a bioactive agent that inhibit or ameliorate the formation of tissue adhesions, such as tissue plasminogen activator, or a sparingly soluble form of tissue plasminogen activator, such as described in U.S. Pat. No. 5,589,169. The agents can provide the oncotic agent of the irrigation compositions. Such oncotic agents and polymers include without limitation proteins naturally found in plasma, such as albumin, globulin and fibrinogen fractions, plasma extenders such as dextrans, 2-hydroxyethyl starches (Hespan), cyclodextrins, carboxymethyl cellulose, polyethylene glycols, glycogens and pluronic acids. Where the preferred amount of polymeric agent increase solution viscosity beyond that preferred for use in irrigation, the polymer can be added after irrigation, and delivered at a lower flow rate suitable in light of the intracranial pressure constraints on delivery speed.

High Flow Rate Methods

In a preferred aspect of the invention, high flow rates, such as 20 mL/min, 25 mL/min, 30 mL/min, 40 mL/min, 50 mL/min or higher are used to assure removal of products from subarachnoid hemorrhage. Methods of achieving high flow rates are described in Barnitz, U.S. Patent Application No. 60/286,063, filed Apr. 24, 2001.

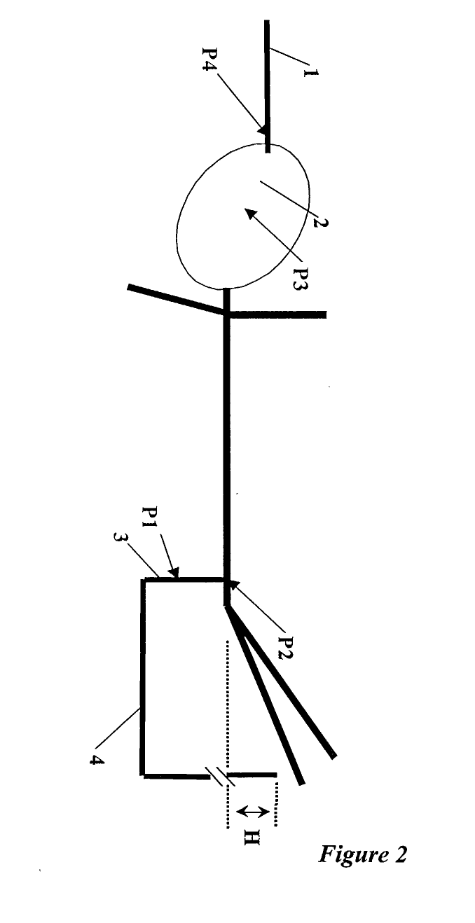

As illustrated in FIG. 2, treatment of a patient begins with the patient in a supine position. Tubing 1 delivers physiologically acceptable liquid (which can be solution, suspension or emulsion) to a ventricular catheter 2, positioned in the lateral ventricle of the brain. Via the aqueduct, cistema magna and subarachnoid spaces, a flow pathway is established to a lumbar outflow catheter 3, positioned for example at an intrathecal space of the lumbar (L4-L5) region of the spine. Any liquid that is physiologically acceptable for the central nervous system (CNS) can be used.

Pressure is monitored at the inlet to the cerebral spinal pathway, P4 (perfusion pressure, pressure at entrance to ventricular catheter), in an intracranial cavity, P3 (intracranial pressure, ICP), and at the outlet, P1 (drainage pressure, pressure at the exit of the lumbar catheter). Pressure in the spinal cord can be measured at P2 (lumbar theca pressure), or that pressure can be inferred from other data and models based on past experience. All pressures are gage values. The outlet tubing 4 can have a spill-over set at a height H (column height) relative to a zero value that is aligned with the approximate center of the spinal column. H is illustrated as at a positive value, but negative values are used after flow rates have been ramped up.

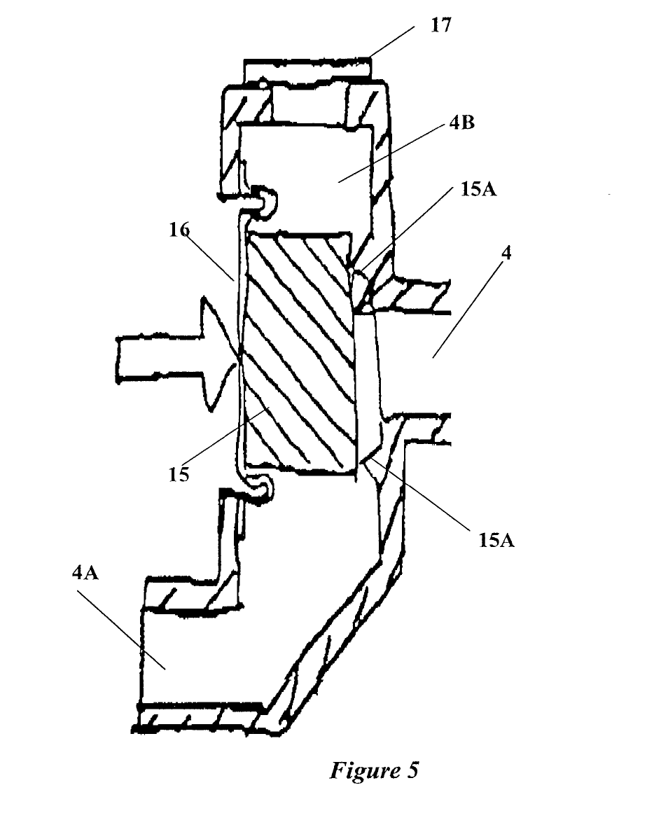

Height H is an illustrative way of setting the outlet pressure P1. Other methods include for example using pressure break devices, actively controlling the input and output pump rates, and maintaining an expansion chamber (bellows) in the outlet tubing for which the expansion, and hence the pressure, can be actively managed. One illustrative pressure break device is illustrated in FIG. 5. Outlet tubing 4 is blocked, when the break pressure has not been obtained, by barrier piece 15, which seats on rim 15A. Rolled diaphragm 16 maintains liquid isolation. The break pressure is applied on the axis indicated by the arrow, and can be set by any of a number of mechanisms known in the art, such as spring-loaded tensioning devices, electromechanical pushing devices, hydraulic systems pressured by pumps or electromechanical pushing devices, gas pressure, and the like. In the illustrated break device, sterile filter 17 allows for gas (e.g., air) intake to manifold 4B, which is connected (independent of barrier piece 15) to outlet tubing 4A. To allow for negative pressure, the pressure break device can be positioned sufficiently below the H=0 level so that easing the break pressure effectively brings P1 to an appropriate negative value. Or, sufficient pumping can be applied to the fluid in outlet tubing 4A (in the absence of a gas intake) to maintain the desired negative pressure. Pressure control can be through active relative control of pumps (e.g., using the feedback loops and controller discussed below) or manual.

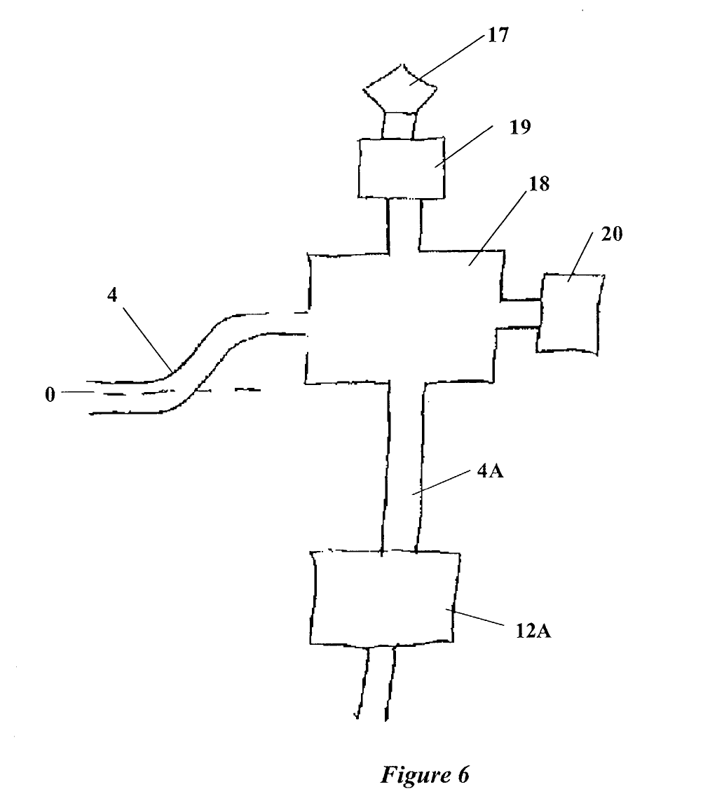

Another illustration of a pressure control device is found in FIG. 6. Manifold 18 is rigid, and can thus maintain a partial reduced pressure (measured against atmospheric). Manifold 18 is preferably placed above (e.g., 5 cm, measured from the bottom of the manifolds connection to tubing 4) the H=0 level. Valve 19 (if present) controls any introduction of gas into manifold 18, and can be for example a variable resistance valve or an adjustable pressure relief valve. Pressure monitor 20 can be a pressure transducer. Recycle pump 12A is suitable to create a reduced pressure in manifold 18. Preferably, pressure control is by active feedback control of recycle pump 12A, based on pressure data, for example from pressure monitor 20.

After initial setup of the catheters, the above introduced parameters can be, with no flow, for example:

| Body Position | H | P1 | P2 | P3 | P4 |
|---|---|---|---|---|---|
| Horizontal | +5 cm | 4 mmHg | 4 mmHg | 4 mmHg | 4 mmHg |

The values after initiation of flow are as set forth below for various flow rates. These values are based on the use of a 14 gauge catheter as the lumbar catheter. Exemplary catheters are described, for example, in co-pending Ser. No. 09/382,136, filed Nov. 26, 1999. The flow resistance of this catheter is a major determinant of P2, and consequently of P3. The use of catheters of different flow resistances will modify the pressure relationships as can be determined with appropriate calculations and modeling. When flow is at 10 mL/min:

| Body | H | P1 | P2 | P3 | P4 |
|---|---|---|---|---|---|
| Position | (cm) | (mmHg) | (mmHg) | (mmHg) | (mmHg) |
| Horizontal | +5 | 4 mmHg | 12 mmHg | 16.5 mmHg | 19.0 mmHg |
| Horizontal | 0 | 0 mmHg | 8 mmHg | 12.5 mmHg | 15.0 mmHg |
| Horizontal | −5 | −4 mmHg | 4 mmHg | 8.5 mmHg | 11.0 mmHg |

When flow is at 20 mL/min:

| Body | H | P1 | P2 | P3 | P4 |
|---|---|---|---|---|---|
| Position | (cm) | (mmHg) | (mmHg) | (mmHg) | (mmHg) |
| Horizontal | 0 | 0 | 16.75 | 27.0 | 32.25 |
| Horizontal | −10 | −8.0 | 8.75 | 19.0 | 24.25 |
| Horizontal | −15 | −12.0 | 4.75 | 15.0 | 20.25 |
| Horizontal | −20 | −16.0 | 0.75 | 11.0 | 16.25 |

When flow is at 30 mL/min:

| Body | H | P1 | P2 | P3 | P4 |
|---|---|---|---|---|---|
| Position | (cm) | (mmHg) | (mmHg) | (mmHg) | (mmHg) |
| Horizontal | −10 | −8.0 | 19.75 | 35.75 | 45.25 |
| Horizontal | −20 | −16.0 | 11.75 | 27.25 | 37.25 |
| Horizontal | −30 | −24.0 | 3.75 | 19.75 | 29.25 |

The shaded values are to be avoided. P2 values of less than about 3.5 are typically avoided.

The central nervous system (CNS) physiologically acceptable liquid used in the above example is a fluorocarbon nutrient emulsion containing eight a constituent compositions is as set forth in the table below for a 1200 mL unit of the emulsion. However, as mentioned, any CNS physiologically acceptable fluid can be used with this invention.

| | Amount |
|---|---|
| Constituent Compositions | g/unit |
| t-Bis-perfluorobutyl ethylene | 200 |
| NaCl, USP | 7.63 |
| NaHCO₃, USP | 2.19 |
| Purified egg yolk phospholipid, | 13.8 |
| KCl, USP | 0.23 |
| MgCl₂-6H₂O, USP | 0.24 |
| CaCl₂-2H₂O, USP | 0.18 |
| Dextrose, USP | 1 |
| Albumin (Human), USP | 20 |
| L-lysine HCl, USP | 0.0032 |
| L-alanine, USP | 0.0034 |
| L-serine, USP | 0.0030 |
| L-threonine, USP | 0.0036 |
| L-arginine, USP | 0.0022 |
| L-leucine, USP | 0.0015 |
| L-isoleucine, USP | 0.0006 |
| L-valine, USP | 0.0020 |
| L-phenylalanine, USP | 0.0010 |
| L-tyrosine, USP | 0.0010 |
| L-histidine, USP | 0.0012 |
| L-methionine, USP | 0.0003 |
| NaH₂PO₄, USP | 4.1 |
| Na₂HPO₄, USP | 0.61 |
| α-ketoglutaric acid | 0.030 |
| Sterile Water for Injection, USP | 1040 mL |

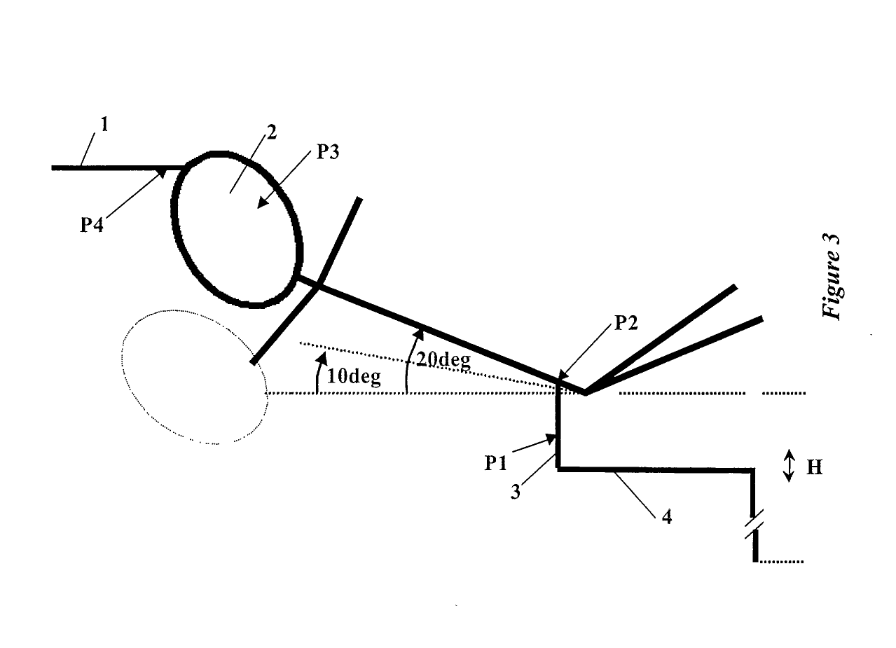

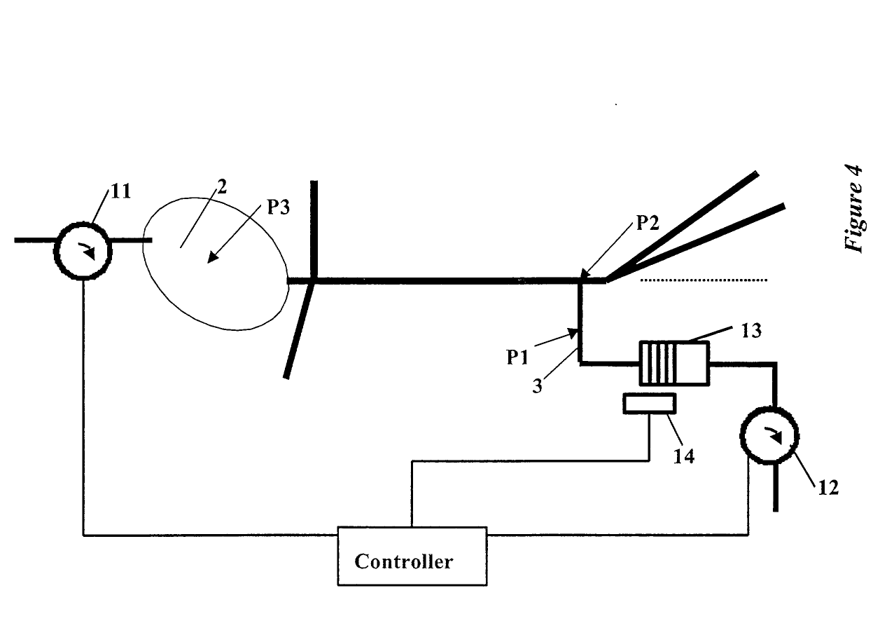

FIG. 3 shows elements of FIG. 2 in a more schematic fashion. After higher flow has been initiated, the patient can be elevated as indicated in FIG. 4. For example, the patient can be safely inclined when flow rates have become high, such as 20 mL/min, 25 mL/min, 30 mL/min or higher. In FIG. 4, the patient is illustrated at a 20 degree incline, with a 10 degree incline illustrated in dotted lines. Incline angles are often in the lower range of, for example, 10 or 20 degrees, but higher inclinations can be used to achieve still more elevated flow rates, such as 50, 60 or 70 mL/min.

Exemplary pressure parameters with an incline are illustrated below. Body position only affects ICP (P3) and perfusion pressure (P4), lumbar theca(P2) and drainage(P1)pressures are unaffected. A five degree incline will reduce ICP and PP by 3.75 mmHg for an average sized patient, by 7.25 mmHg for a 10 degree incline, by 11.0 mmHg for a fifteen degree incline, and by 14.50 mmHg for a twenty degree incline. For example, when flow is at 30 mL/min:

| Body | H | P1 | P2 | P3 | P4 |
|---|---|---|---|---|---|
| Position | (cm) | (mmHg) | (mmHg) | (mmHg) | (mmHg) |
| Horizontal | −30 | −24.0 | 3.75 | 19.8 | 29.3 |
| 5° incline | −30 | −24.0 | 3.75 | 16.0 | 25.5 |
| 10° incline | −30 | −24.0 | 3.75 | 12.5 | 22.0 |

In another aspect, the delivery algorithm takes into account a phenomenon (and risk) involved in recycling the liquid that has cycled through the cerebral spinal pathway back through the pathway. A mismatch in inflow and outflow rates can occur, resulting from the tolerances in the two pumping systems, a difference in CSF production and absorption, or a change in ICP and the concurrent change in CNS volume due to compliance in the CNS. Such a mismatch could lead to an over or under pressure condition in the patient. This risk is addressed in one or more of two ways.

First, the flow rate of pumping of the effluent is maintained a rate sufficiently higher than the delivery flow rate to account for these sources of variation. A gas/air intake (preferably fitted with a sterile filter) in the effluent line provides a fluid source to account for the higher flow rate. The intake is linked to the tubing/plumbing before the pump inlet. Before recycle, the liquid can be passed through a holding container in which the extra gas is separated away (preferably through a sterile filter). This format is effective to not, of itself, create a significant negative pressure. The minor pressure differential across the sterile filter is not a significant pressure. A device for accomplishing these functions with peristaltic pumps is described in copending U.S. Application No. 60/286,057, filed Apr. 24, 2001. A preferred set-point in the flow differential is between 5 and 15%, such as about 10%. Note, that this is the differential set with respect to the average calibrated flow rate, but in some instances the differential is established in part in acknowledgement that the pumps used for reliable, non-pulsatile, sterile pumping can be somewhat variable in their actual flow rate.

Second, as illustrated in FIG. 5, a bellows 13 is incorporated into the tubing/plumbing before the recycle pump 12, and the expansion or contraction of the bellows is monitored by monitor 14. Monitor 14 sends data to the controller, which adjusts the rate of delivery pump 11 or recycle pump 12 as appropriate. Data from pressure monitoring devices can also be sent to the controller, so that the controller can avoid increasing the flow of delivery pump 11, or reduce the flow of delivery pump 11, in response to pressure data.

The monitor 14 can be physically connected to the bellows via a linear transducer or linear potentiometer, providing an electrical signal for the amount of movement in the bellows. Or, the monitor can monitor the offset of the below with a light reflectance angle, with multiple reflectance monitors that indicate whether the bellows is within or without a reflectance pathway, by measuring the distance analog of an acoustic reflectance. Other methods recognized in the art for measuring displacements can be used. Where a bellows such as illustrated functions to control pressure at the drainage end, the same devices for controllably applying pressure as discussed above with reference to the break pressure can be used to exert the required force on the bellows.

### EXAMPLE 1

A patient with aneurysmal subarachnoid hemorrhage is perfused within 6 hours after completion of repair of the cerebral aneurysm. The patients have a ventricular perfusion catheter and a lumbar exit catheter placed. A second ventriculostomy on the ipsilateral hemisphere is performed to allow measurement of brain temperature and pressure using the commercially available pressure sensor, such as a Codman ICP Sensor or a Licox® intraparenchymal pressure, temperature and oxygen sensor. The exit route is via a lumbar catheter. Patients are perfused with the fluid of example 2 at a rate of 5-60 mL/min via the ventriculo-lumbar or ventricle-ventricle route for up to 72 hours. Patients receive standard intensive care throughout the perfusion period. After perfusion is complete the catheters are removed.

### EXAMPLE 2

A patient with aneurysmal subarachnoid hemorrhage is perfused within 6 hours after completion of repair of the cerebral aneurysm. The patients have a ventricular perfusion catheter and a ventricular exit catheter placed. A second burr hole on the ipsilateral hemisphere would be performed to allow measurement of brain temperature and pressure using a commercially available pressure sensor, such as a Codman ICP Sensor or a Licox® intraparenchymal pressure, temperature and oxygen sensor. Patients are perfused with an ACSF of example 3 at a rate of 5-80 mL/min via the ventriculo-lumbar or ventricle-ventricle route for up to 72 hours. Patients receive standard intensive care throughout the perfusion period. After perfusion is complete the catheters are removed.

### EXAMPLE 3

Alternating ACSF and ACSF Plus Thrombolytic

Patient with aneurysmal subarachnoid hemorrhages are perfused within 3 hours after completion of repair of the cerebral aneurysms. The patients have a ventricular perfusion catheter and a ventricular exit catheter placed. For each, a second burr hole on the ipsilateral hemisphere is placed to allow measurement of brain temperature and pressure using a commercially available pressure sensor, such as a Codman ICP Sensor or a Licox® intraparenchymal pressure, temperature and oxygen sensor.

Patients are perfused with an ACSF of examplary formulation 3 alternating, at least once during 24 hours, with ACSF of examplary formulation 22. Patients are first perfused with an ACSF of examplary formulation 3 at a rate of 2-20 mL/min via the ventriculo-lumbar or ventricle-ventricle route for 3 hours. Patients are then perfused with 400 mL an ACSF of examplary formulation 22 (ACSF plus tPA) at a rate of 2-10 mL/min for up to 3 hours. If for a patient 400 mL of ACSF is totally perfused before 3 hours have elapsed, then the perfusion is stopped and the ACSF plus tPA allowed to remain in the patient for the 3 hour period. At the end of the 3 hours, perfusion with ACSF of examplary formulation 3 is again started at a rate of 2-20 mL/min for 18 hours.

Patients receive standard intensive care throughout the perfusion period. After perfusion is complete the catheters are removed.

Definitions

The following terms shall have, for the purposes of this application, the respective meanings set forth below.

amount effective to reduce swelling. An amount of serum albumin effective to reduce swelling of neurologic tissue at flow rates through the cerebrospinal pathway in excess of 2 mL/min is an amount that would, in the absence of other oncotic agents, allow such flow rates without medically contraindicated amounts of swelling. The amount is preferably sufficient to be effective in reducing swelling at flow rates of 5 mL/min or, more preferably, 10 mL/min.

cerebrospinal tissue. Cerebrospinal tissue includes all tissues bathed sufficiently to allow exchange of metabolites and nutrients by cerebrospinal fluid. Cerebrospinal tissue includes the membranes of the meninges.

effective amount. The meaning of “effective amount” will be recognized by clinicians but includes an amount effective to reduce, ameliorate or eliminate one or more symptoms of the disease sought to be treated or the condition sought to be avoided or treated, or to otherwise produce a clinically recognizable change in the pathology of the disease or condition. An effective amount of an emulsifying agent shall include an amount effective to more rapidly reduce the level of an indication of blood from the effluent from irrigating the region of the hemorrhage than would occur in the absence of the emulsifying agent. Where the emulsifying agent plays a dual role of emulsifying a component of the synthetic cerebrospinal fluid, the comparative synthetic cerebrospinal fluid lacks the component to be emulsified.

oncotic agent. By oncotic agent is meant substances, generally macromolecules, that are of a size that is not readily able to leave the body cavity or other fluid containing body spaces (such as the cerebrospinal pathway, including the cerebral ventricles and subarachnoid spaces) into which they are inserted. Such oncotic agents are exemplified by blood plasma expanders which are known in general as macromolecules having a size sufficient to inhibit their escape from the blood plasma through the circulatory capillary bed into the interstitial spaces of the body. Serum albumin, preferably human serum albumin, is one well known blood plasma protein that can be used as an oncotic agent. Polysaccharide blood plasma expanders are often glucan polymers. For example, Hetastarch (a product of American Home Products) is an artificial colloid derived from a waxy starch composed almost entirely of amylopectin with hydroxyethyl ether groups introduced into the alpha (1-4) linked glucose units. The colloid properties of a 6% solution (wt/wt) of hetastarch approximate that of human serum albumin. Other polysaccharide derivatives may be suitable as oncotic agents in the blood substitute according to the invention. Among such other polysaccharide derivatives are hydroxymethyl alpha (1-4) or (1-6) polymers and cyclodextrins. In general, it is preferred that the polysaccharide is one that is non-antigenic. High molecular weight agents such as Dextran 70 having a molecular weight of about 70,000 Daltons are generally less preferred because they increase viscosity of the colloidal solution and impair the achievement of high flow rates. Preferably, the oncotic agent is in an amount effective to provide, in conjunction with other components of a fluorocarbon nutrient emulsion or a nutrient solution, an oncotic pressure of one to seven torr.

respiration. Respiration is the physical and chemical processes by which an organism supplies its cells and tissues with the oxygen needed for metabolism and, preferably, relieves them of the carbon dioxide formed in energy-producing reactions.

respiration-supporting amount. A respiration-supporting amount of oxygen is an amount that would, in model experiments, provide a statistically significant reduction in morbidity following a focal ischemic event.

suspending or solubilizing agent for hydrophobic biomaterials. A suspending or solubilizing agent for hydrophobic biomaterials is an emulsifying agent or an agent that dissolves at least certain biomolecules having hydrophobic moieties, such as serum albumin, lipoproteins, and the like. Such agents are preferably physiologically acceptable, meaning that any propensity to disrupt brain or spinal tissue at the concentrations used is sufficiently small so that the benefits of the method described herein outweigh any detrimental effects. As exemplified by serum albumin (preferably human when for use in humans, or the source otherwise matching the species treated), a suspending or solubilizing agent for hydrophobic biomaterials can be an oncotic agent.

Where ranges are given as appropriate or preferred, and narrower ranges are also provided, it should be recognized that any upper preferred value can be paired with any lower preferred value (or vice versa) to define another preferred range. Moreover, if in presenting focused recitations of preferences for one factor in combination with preferences for another factor, it will be recognized that, unless there is a particular contraindication, all combinations of preferences are themselves preferred.

Publications and references, including but not limited to patents and patent applications, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the manner described above for publications and references.

While this invention has been described with an emphasis upon preferred embodiments, it will be obvious to those of ordinary skill in the art that variations in the preferred devices and methods may be used and that it is intended that the invention may be practiced otherwise than as specifically described herein. Accordingly, this invention includes all modifications encompassed within the spirit and scope of the invention as defined by the claims that follow.

What is claimed:

1. A method of treating neurologic hemorrhage comprising:
inserting a inflow catheter and an outflow catheter into a cerebrospinal pathway to create a flow pathway for irrigating the area of the hemorrhage; and
irrigating via the flow pathway the area of the hemorrhage with a synthetic cerebrospinal fluid for a period of time effective to reduce an indication of the presence of blood in effluent from the outflow catheter.

2. The method of claim 1, wherein irrigating is maintained for at least, in aggregate, 6 hours.

3. The method of claim 1, wherein the flow rate is at least 5 mL/min.

4. The method of claim 1, wherein said synthetic cerebrospinal fluid contains an aqueous emulsion of a fluorocarbon.

5. The method of claim 1, wherein said fluorocarbon is bis-perfluorobutyl ethylene (F44E).

6. The method of claim 1, wherein the synthetic cerebrospinal fluid comprises an effective amount of a physiologically acceptable suspending or solubilizing agent for hydrophobic biomaterials.

7. The method of claim 6, wherein the suspending or solubilizing agent is phospholipid.

8. The method of claim 6, wherein the suspending or solubilizing agent is serum albumin.

9. The method of claim 1, further comprising:
administering in the synthetic cerebrospinal fluid an effective amount of a thrombolytic agent, an antioxidant, or a vasodilating agent.

10. The method of claim 1, further comprising:
after the period of time, adding to the synthetic cerebrospinal fluid an effective amount of a thrombolytic agent, an antioxidant, or a vasodilating agent and continuing the perfusion for a second period of time.

11. The method of claim 10, further comprising:
after the second period of time, continuing the perfusion with the synthetic cerebrospinal fluid lacking the added thrombolytic agent, antioxidant, or vasodilating agent.

12. The method of claim 1, further comprising:
monitoring the amount of subarachnoid blood in the cerebrospinal pathway, or in effluent from the irrigating flow; and
ending irrigating flow when the monitored amount reduces below a predetermined value.

13. The method of claim 1, wherein the synthetic cerebrospinal fluid comprises:

| Component | Amount Per Dose | |
|---|---|---|
| Na⁺ | 135-145 | mEq/L |
| HCO₃⁻ | 20-25 | mEq/L |
| P0₄⁻² | 1.2-2.0 | mg/L |
| K⁺ | 2.7-3.9 | mEq/L |
| Mg⁺² | 2.0-2.5 | mEq/L |
| Ca⁺² | 2.0-3.0 | mEq/L |
| Cl⁻ | 115-125 | mEq/L |
| Glucose | 500-1500 | mg/L |
| Albumin | 5-40 | g/L |

14. The method of claim 13, wherein the synthetic cerebrospinal fluid comprises 5-20% w/v of an oxygen-carrying fluorocarbon compound.

15. The method of claim 13, wherein the synthetic cerebrospinal fluid comprises 10 to 30 mg/mL of albumin.

16. The method of claim 13, wherein the synthetic cerebrospinal fluid comprises a nutritional amount of lysine, alanine, serine, threonine, arginine, leucine, isoleucine, valine, phenylalanine, tyrosine, histidine, methionine and α-ketoglutaric acid.

17. The method of claim 1, further comprising,
surgically removing a subarachnoid clot.

18. The method of claim 1, wherein the synthetic cerebrospinal fluid is infused into the subarachnoid space of the brain, and drained from the spinal subarachnoid space.

19. The method of claim 1, wherein the irrigating is initiated prior to, or concurrently with, initiating an operation to repair an aneurysm.

20. The method of claim 17, wherein the synthetic cerebrospinal fluid is maintained at a temperature from 15° C. to 35° C. during the operation.

21. The method of claim 1, wherein the neurologic hemorrhage is IVH, ICH or SCH.

22. A synthetic cerebral spinal fluid comprising:
a physiologically acceptable mixture of salts;
serum albumin in an amount effective to provide an oncotic pressure of one to seven torr;
one or more of (a) a clot-dissolving effective amount of tPA or urokinase or (b) a free-radical formation inhibiting effective amount of ascorbic acid.

23. The synthetic cerebral spinal fluid of claim 22, wherein the serum albumin is human serum albumin.

24. The synthetic cerebral spinal fluid of claim 22, wherein the serum albumin is present in an amount effective to reduce swelling of neurologic tissue at flow rates through the cerebrospinal pathway in excess of 2 mL/min.

25. A method of reducing the occurrence of or ameliorating the severity of neurologic adhesion resulting from a surgery on cerebrospinal tissue or from an inflammation of cerebrospinal tissue comprising:
identifying a subject with a tissue at risk for forming a neurologic adhesion resulting from a surgery on cerebrospinal tissue or from an inflammation of cerebrospinal tissue;
inserting a inflow catheter and an outflow catheter into a cerebrospinal pathway to create a flow pathway for irrigating the tissue; and
irrigating via the flow pathway the tissue with a synthetic cerebrospinal fluid,
wherein, in the case of surgery the synthetic cerebrospinal fluid does not have a respiration-supporting amount of oxygen, and
wherein the inflammation of cerebrospinal tissue does not result from stroke.

26. The method of claim 25, wherein irrigating is maintained for at least, in aggregate, 6 hours.

27. The method of claim 25, wherein the flow rate is at least 5 mL/min.

28. The method of claim 25, wherein said synthetic cerebrospinal fluid contains an aqueous emulsion of a fluorocarbon.

29. The method of claim 25, wherein said fluorocarbon is bis-perfluorobutyl ethylene (F44E).

30. The method of claim 25, wherein the synthetic cerebrospinal fluid comprises an effective amount of a physiologically acceptable suspending or solubilizing agent for hydrophobic biomaterials.

31. The method of claim 30, wherein the suspending or solubilizing agent is phospholipid.

32. The method of claim 30, wherein the suspending or solubilizing agent is serum albumin.

33. The method of claim 25, further comprising:
administering in the synthetic cerebrospinal fluid an effective amount of a thrombolytic agent, an antioxidant, or a vasodilating agent.

34. The method of claim 25, wherein the synthetic cerebrospinal fluid comprises:

| Component | Amount Per Dose | |
|---|---|---|
| Na⁺ | 135-145 | mEq/L |
| HCO₃⁻ | 20-25 | mEq/L |
| P0₄⁻² | 1.2-2.0 | mg/L |
| K⁺ | 2.7-3.9 | mEq/L |
| Mg⁺² | 2.0-2.5 | mEq/L |
| Ca⁺² | 2.0-3.0 | mEq/L |
| Cl⁻ | 115-125 | mEq/L |
| Glucose | 500-1500 | mg/L |
| Albumin | 5-40 | g/L |

35. The method of claim 34, wherein the synthetic cerebrospinal fluid comprises 5-20% w/v of an oxygen-carrying fluorocarbon compound.

36. The method of claim 34, wherein the synthetic cerebrospinal fluid comprises 10 to 30 mg/mL of albumin.

37. The method of claim 34, wherein the synthetic cerebrospinal fluid comprises a nutritional amount of lysine, alanine, serine, threonine, arginine, leucine, isoleucine, valine, phenylalanine, tyrosine, histidine, methionine and α-ketoglutaric acid.

39. The method of claim 25, wherein the synthetic cerebrospinal fluid is infused into the subarachnoid space of the brain, and drained from the spinal subarachnoid space.

40. The method of claim 39, wherein the synthetic cerebrospinal fluid is maintained at a temperature from 15° C. to 35° C. during the operation.
